(19) United States
(12) Patent Application Publication
(10) Pub. No.: US 2018/0243537 A1
(43) Pub. Date: Aug. 30, 2018

(54) SYSTEM FOR SECURING MULTI-LUMEN MEDICAL ARTICLES

(71) Applicant: 3M INNOVATIVE PROPERTIES COMPANY, St. Paul, MN (US)

(72) Inventors: Naimul Karim, Maplewood, MN (US); Jennifer N. Hanson, Woodbury, MN (US); Shilpi K. Sanghi, Stanford, CA (US); Jia Hu, Mounds View, MN (US)

(21) Appl. No.: 15/755,942

(22) PCT Filed: Nov. 9, 2016
(86) PCT No.: PCT/US2016/061089

§ 371 Date: Feb. 27, 2018

Related U.S. Application Data
(60) Provisional application No. 62/255,556, filed on Nov. 16, 2015.

(51) Int. Cl. A61M 25/02

(52) U.S. Cl. CPC A61M 25/02; A61M 2025/0266

(57) ABSTRACT

A securement system for securing a multi-lumen medical article. The system can include a patch, and a longitudinal flap. The flap can include a fixed proximal portion that is fixed with respect to the patch; a hinge; and a free distal portion movable via the hinge with respect to the patch and the fixed proximal portion of the flap between an open position and a closed position. The hinge can include a lateral width that is less than a lateral width of the patch and less than a lateral width of the flap. The hinge can be dimensioned to be received between two adjacent proximal tubings of the medical article, e.g., to provide a longitudinal stop to the medical article. The free distal portion can be configured to secure at least the multi-lumen joint of the medical article to the patch.

### FIELD

The present disclosure relates to a securement system for safely and reliably securing a multi-lumen medical article, such as medical tubing, upon a desired location of a patient's body.

### BACKGROUND

Various medical treatments often require the use of medical articles, and particularly, medical tubing. In many cases the medical article must be secured to a patient's body. For example, it can be necessary to introduce fluids and liquid medications directly into a blood vessel of a patient. For short term general use, a peripheral intravenous (IV) catheter can be placed onto a patient's arm. For longer term and more specialized needs, central line catheters or other devices are used. In another example, a urinary catheter (such as a Foley catheter) may be necessary for draining urine from a patient's bladder.

Healthcare providers often secure catheters or other medical articles to patients during hospital stays or in-home care. Securing the medical article (e.g., medical tubing) aids in proper positioning, which prevents dislodgement or tangling and which may cause leakage or interruptions in medication dosing. Securement of such articles also minimizes patient discomfort, and reduces risk for infection.

In order to keep medical articles properly positioned for the duration of treatment, the medical article may be secured to the patient in a variety of ways. Some existing securement devices are generally designed for a specific type or size of medical article (e.g., medical tubing, catheter, etc.). As a result, multiple securement devices may be needed to accommodate different types or sizes of medical articles and/or tubing, e.g., in hospitals and clinical settings. This can add to the cost and complexity of sourcing, inventory, storage, and selection of the securement devices. Additionally, many securement devices still suffer effects of patient movement in which tubing may become kinked or pinched and restrict flow of medication, blood, or urine. Furthermore, many securement devices include large, bulky, rigid hardware elements that are not conformable to a patient's body and which can increase patient discomfort, and can cause pressure ulcers, while also providing large relief structures with a greater risk for disruption or entanglement.

There remains a need for securement devices that can be comfortably worn for longer periods of time, and that accommodate varying sizes of medical articles or tubing, while allowing for patient movement without disruption or kinking of the tubing.

### SUMMARY

The present disclosure generally relates to a securement system comprising a longitudinal flap (e.g., a tape flap) and a patch for safely and reliably securing a multi-lumen medical article, such as medical tubing (e.g., a catheter system), upon a desired location of a patient's body. In general, multi-lumen medical articles of the present disclosure can include a multi-lumen hub (or joint), at least two proximal tubings connected to the hub, and at least one distal tubing connected to the hub.

Some aspects of the present disclosure provide a securement system for securing a multi-lumen medical article, the medical article comprising a multi-lumen joint, at least two proximal tubings connected to the multi-lumen joint and at least one distal tubing connected to the multi-lumen joint. The system can include a longitudinal direction, a patch, and a longitudinal flap. The patch can include a first major surface configured to receive at least a portion of the medical article, and a second major surface, opposite the first major surface, comprising a skin-contact adhesive. The longitudinal flap can include a first major surface, and a second major surface, opposite the first major surface, configured to be coupled to at least a portion of the medical article and the first major surface of the patch. The flap can further include a fixed proximal portion that is fixed with respect to the patch; a hinge; and a free distal portion movable via the hinge with respect to the patch and the fixed proximal portion of the flap between an open position and a closed position. The hinge can be located within a perimeter of the patch, such that the hinge does not extend to or form a portion of the perimeter of the patch, and the hinge can include a lateral width that is less than a lateral width of the patch and less than a lateral width of the flap. The hinge can be dimensioned to be received between two adjacent proximal tubings of the medical article, e.g., to provide a longitudinal stop to the medical article. The free distal portion can be configured to overlap (e.g., in the closed position) at least the multi-lumen joint of the medical article and at least a portion of the patch to secure at least the multi-lumen joint of the medical article to the first major surface of the patch.

Other features and aspects of the present disclosure will become apparent by consideration of the detailed description and accompanying drawings.

### BRIEF DESCRIPTION OF THE DRAWINGS

FIG. 1A is a perspective view of a securement system according to one embodiment of the present disclosure, the system comprising a patch and a longitudinal flap, the longitudinal flap having cutaway regions, the system shown with a medical article unsecured.

FIG. 1B is a perspective view of an alternative embodiment of the system of FIG. 1A.

FIG. 2 is a top plan view of the securement system of FIG. 1A, illustrating a hinge and cutaway regions of the flap, the system shown with the medical article secured.

FIGS. 3A-3F illustrate top plan views of alternative cutaway regions of flaps of systems of the present disclosure.

FIG. 4A is a schematic side cross-sectional view of the system of FIGS. 1A and 2, with the flap shown in a first, unsecured position.

FIG. 4B is a schematic side cross-sectional view of an alternative embodiment of the system of FIGS. 1A, 2 and 4A, with the flap shown in a first, unsecured position.

### DETAILED DESCRIPTION

The present disclosure generally relates to universal securement systems for securely and reliably securing multi-lumen medical articles. Generally, the systems of the present disclosure offer a unique alternative to systems comprising complex, rigid couplings or hardware that tend to be product-specific. In general, systems of the present disclosure include a patch that adheres to the patient's skin, and a longitudinal flap having (i) a fixed proximal portion that is fixed with respect to the patch, (ii) a hinge located within a perimeter of the patch and having a lateral width that is less than a lateral width of the patch and less than a lateral width of the flap, and (iii) a free distal end that is movable via the hinge with respect to the patch and the fixed proximal portion of the flap. The hinge can be dimensioned to be received between two adjacent proximal tubings (e.g., to abut a bifurcation point) of a multi-lumen medical article to provide at least a soft longitudinal stop for the medical article.

The present disclosure generally relates to securement systems and methods for safely and reliably securing a multi-lumen medical article, such as medical tubing, upon a desired location of a patient's body. The securement systems can be universal to accommodate and reliably secure a large variety of medical articles or class of medical articles (e.g., Foley catheters and peripherally inserted central catheters (PICCs)), and can be particularly useful for securing multi-lumen medical articles that need to be secured to a patient over a prolonged period of time, such as weeks or months.

Examples of multi-lumen medical articles that can be employed with the medical article securement devices and systems of the present disclosure include, but are not limited to, medical tubing or fluid supply lines, other similar articles, or combinations thereof. Examples of medical tubing can include, but are not limited to urinary catheters (e.g., Foley catheters), intravenous (IV) catheters, central venous catheters (CVCs), peripherally inserted central catheters (PICCs), arterial catheters, chest tubes, drainage tubes, infant umbilical catheters, and dialysis catheters.

### Definitions

The term “a”, “an”, and “the” are used interchangeably with “at least one” to mean one or more of the elements being described.

The term “and/or” means either or both. For example “A and/or B” means only A, only B, or both A and B.

The terms “including,” “comprising,” or “having,” and variations thereof, are meant to encompass the items listed thereafter and equivalents thereof as well as additional items.

Unless specified or limited otherwise, the term “coupled” and variations thereof are used broadly and encompass both direct and indirect couplings.

Relative positional terms, such as “top,” “upper,” “lower,” and the like, are only used to describe elements as they relate to one another, but are in no way meant to indicate or imply necessary or required orientations of the apparatus, or to specify how the invention described herein must be used, mounted, displayed, or positioned in use.

The terms “longitudinal” and “axial” are used to refer to a direction or axis that is generally parallel to the direction in which the medical article extends and generally parallel to the overall direction of fluid flow, e.g., along a catheter line.

The term “lateral” is used to refer to a direction or axis that is perpendicular to the longitudinal axis or direction and is used to represent side-to-side motion of a medical article.

The terms “vertical” and “normal” are used to refer to a direction or axis that is normal to both the longitudinal and lateral directions or axes, as well as to the surface of a patient's skin when the medical article securement system is coupled to the patient's skin, and is used to represent the direction of motion toward and away from the skin surface.

The term “proximal” and “distal” are used to represent relative axial directions, relative to a medical practitioner operating or holding the medical article. That is, the term “distal” is used to refer to the direction away from the medical practitioner (and toward an insertion site on the patient and inside the patient's body), and the term “proximal” is used to refer to the direction toward the medical practitioner (and toward the outside of the patient's body, away from the insertion site). For example, the distal end of a catheter is inserted into the patient, while the proximal end extends exterior of the patient toward the medical practitioner. The distal end of the medical article securement system refers to the end of the system that is configured to be oriented toward the distal end of the medical article to which it will be coupled, and the proximal end of the medical article securement system refers to the end of the system that is configured to be oriented toward the proximal end of the medical article.

The terms “layer,” “sheet,” and “dressing,” or variations thereof, are used to describe an article having a thickness that is small relative to its length and width.

The term “repositionable” refers to the ability of an article or surface to be, at least initially, repeatedly coupled to (e.g., adhered to) and removed from a surface or substrate without substantial loss of coupling capability (e.g., adhesion) and without damage to either surface (e.g., article or underlying substrate) being coupled together. By way of example, some pressure-sensitive adhesives and mechanical fasteners are repositionable.

The phrase “mechanical fastener” or “touch fastener” generally refers to a fastener that includes two mating, or engagement, surfaces configured to be applied to one another, each mating surface having a plurality of engagement structures or features, such that engagement structures on one mating surface are configured to engage with the engagement structures on the opposing mating surface. In some embodiments, the mechanical fastener can include two flexible mating strips or layers. In some embodiments, the mechanical fastener can include a first mating surface comprising tiny, stiff protrusions shaped like hooks that are configured to engage a second mating surface comprising pliable loops (i.e., a “hook and loop fastener,” or “hook and pile fastener”). In some embodiments, the mechanical fastener can include inter-engaging hooks (e.g., self-engaging hooks) on both mating surfaces (i.e., a “hook and hook fastener” or a “self-engaging hook fastener”).

“Peel force” refers to the force needed to “peel” one surface from another surface at an angle with respect to the plane between the surfaces. Adhesive peel force can be measured using ASTM D3330/D3330M-04 (2010). Peel force between mating surfaces of a mechanical fastener can be measured using ASTM D5170-98 (2015)—Standard Test Method for Peel Strength (“T” Method) of Hook and Loop Touch Fasteners.

“Shear strength” (or “shear force”) refers to the resistance to forces that cause, or tend to cause, two contiguous parts of a body to slide relatively to each other in a direction parallel to their plane of contact. That is, shear strength is the amount of force required to move one surface relative to another surface when the two surfaces are pulled in opposite directions parallel to their plane of contact. Adhesive shear force can be measured using ASTM D3654M-06 (2011). Shear force between mating surfaces of a mechanical fastener can be measured using ASTM D5169-98 (2015)—Standard Test Method for Shear Strength (Dynamic Method) of Hook and Loop Touch Fasteners.

The term “flexible” can generally be used to refer to a material that is drapable. That is, a section of material 5 cm×15 cm when held upright (long end up) folds over under its own weight to drop the opposite end to or below the holder, when performed at ambient conditions. The term “rigid” can generally be used to refer to a material that is essentially non-drapable. That is, a section of material 5 cm×15 cm when held upright (long end up) stands straight up with little or no deflection, when performed at ambient conditions. In some embodiments, rigid materials can show less than 20 degrees of deflection from vertical. “Semi-rigid” materials can be those that exhibit more than 20 degrees of deflection but whose opposite end does not drop below the holder.

The securement systems of the present disclosure include one or more flaps (i.e., flexible flaps) oriented substantially longitudinally, with a hinge that has a lateral width that is less than that of the patch and the flap itself, such that the narrow hinge can be positioned between two proximal tubings entering a multi-lumen joint and can provide a longitudinal stop to the medical article without requiring rigid, bulky components. While some embodiments can further include a post or a small rigid component on top of or otherwise adjacent the hinge to further aid in inhibiting longitudinal movement of the medical article, systems of the present disclosure can include mostly soft, flexible, and comfortable components, resulting in low-cost, universal securement systems.

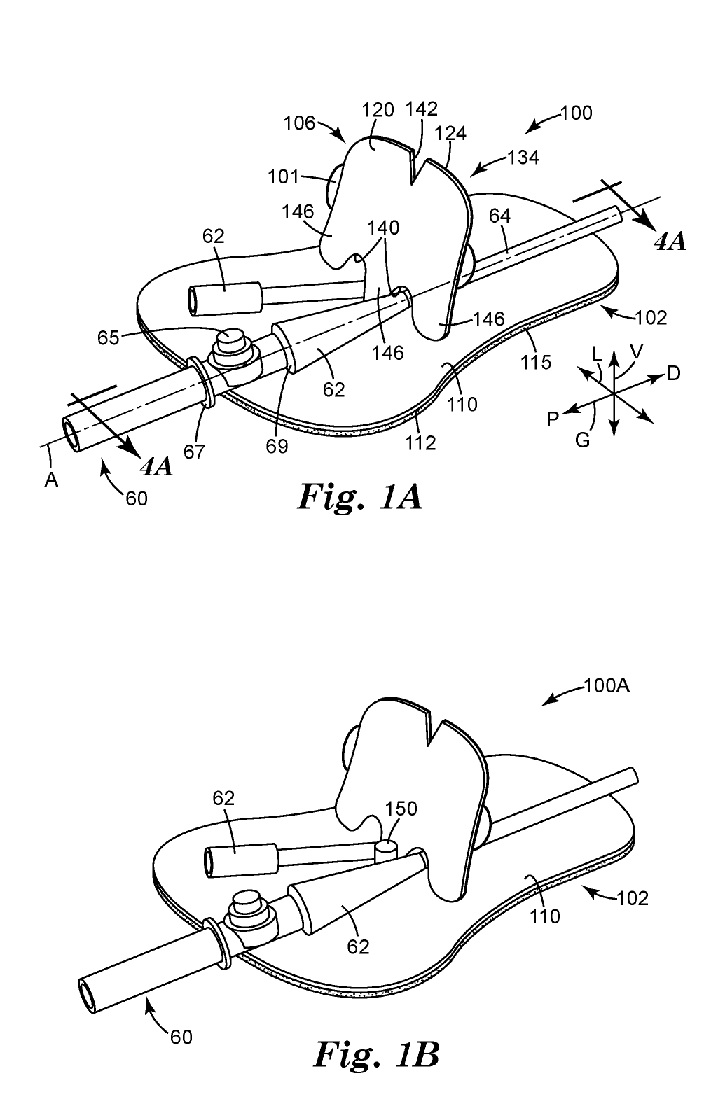

FIGS. 1A, 2 and 4A illustrate a securement system 100 according to one embodiment of the present disclosure. FIGS. 1B, 3A-3F and 4B illustrate various alternative features of the system 100.

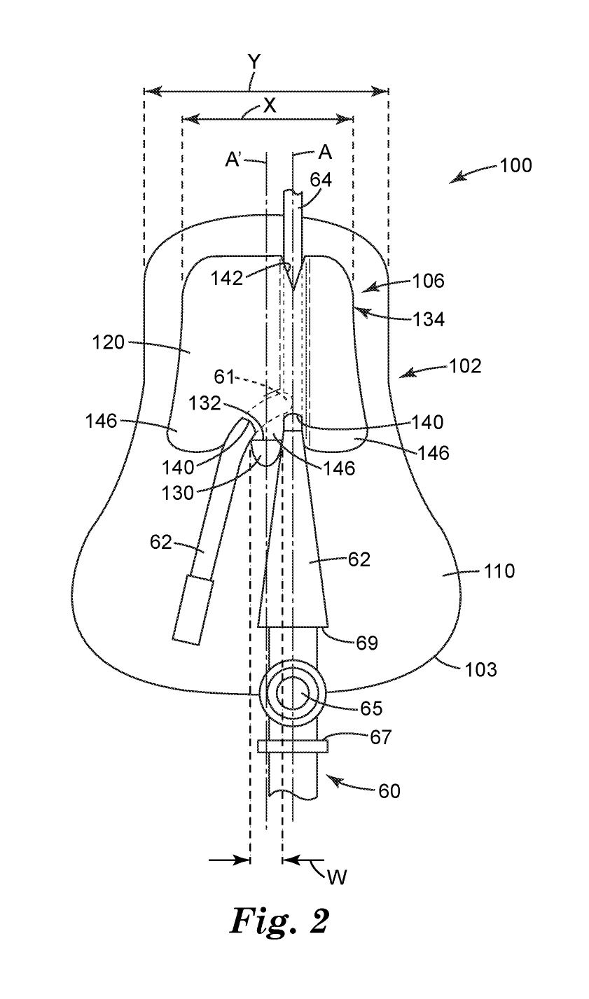

With references to FIGS. 1A, 2 and 4A, the system 100 is shown as securing a medical article 60, which is shown by way of example as being a multi-lumen medical tubing, and particularly, as being a Foley catheter. The medical article 60 includes a multi-lumen joint (or hub) 61 (see FIGS. 2 and 4A), at least two proximal tubings 62 connected to (i.e., each comprising a lumen that is fluidly coupled to) the multi-lumen joint 61 and at least one distal tubing 64 connected to (i.e., comprising a lumen that is fluidly coupled to) the multi-lumen joint 61 (e.g., such that the multi-lumen joint 61 includes at least a bifurcation, but could also include a trifurcation, etc.).

The medical article 60 has a longitudinal axis A. The system 100 includes a longitudinal direction G configured to be oriented substantially aligned with or parallel to the longitudinal axis A of the multi-lumen medical article 60. More specifically, the longitudinal direction G can include a proximal (longitudinal) direction P and a distal (longitudinal) direction D. The system 100 has a longitudinal axis A′ (see FIG. 2) that is configured to be substantially aligned with, or parallel to, the longitudinal axis A of the medical article 60 when the medical article 60 is positioned on or coupled to the system 100.

As shown in FIGS. 1A, 2 and 4A, the system 100 can include a patch (or base dressing, or base layer) 102 configured to receive the medical article 60, and one or more longitudinal flaps 106. The system 100 is shown as including only one longitudinal flap 106; however, other embodiments of systems of the present disclosure include multiple flaps. When describing the system 100, one flap 106 will be described for simplicity, but it should be understood that the description can apply to as many flaps that are employed in a given system.

The flap 106 can be coupled to the patch 102 and/or integrally formed with the patch 102, and the patch 102 can be adhered to skin. The flap 106 can be used to secure the medical article 60 to the system 100 (i.e., to the patch 102) and the patient's skin. As shown in FIGS. 1A, 2 and 4A, the flap 106 can be positioned with respect to the patch 102 and oriented with respect to the longitudinal direction G, such that the flap 106 opens and closes longitudinally (i.e., is a longitudinal flap), and particularly, is fixed proximally and flaps closed distally. The flap 106 can secure the medical article 60 in such a way that it inhibits movement of the medical article 60 in the longitudinal direction G and in a vertical direction V, and also can further inhibit movement of the medical article 60 in a lateral direction L.

In some embodiments, the flap 106 and the patch 102 can be formed of the same backing material. In some embodiments, the flap 106 may be formed of a different backing material than the patch 102. Various additional details regarding backings of the present disclosure are described in greater detail below under the section entitled, “Backings.”

The patch 102 includes a first major surface 110 configured to face away from the patient's skin, and a second major surface 112 opposite the first major surface 110 that comprises a skin-contact adhesive 115 for adhering to the skin. Although the patch 102 is illustrated as having rounded edges and having a larger proximal end than distal end, it should be understood that the patch 102 can take on a variety of shapes and sizes, depending on the shapes and configurations of the other elements of the system 100 and the medical article 60 to be coupled to the system 100. In some embodiments, the patch 102 comprises a laminated structure comprising one or more of a fabric, a woven fibrous web, a nonwoven fibrous web, a knit, a polymeric film, or combinations thereof.

The skin-contact adhesive 115 is generally a pressure-sensitive adhesive, and particularly is a pressure-sensitive adhesive that is capable of securely but releasably adhering or bonding to skin (e.g., mammalian skin). The skin-contact adhesive 115 is also generally safe and non-toxic. Skin-contact adhesive layers will generally be selected according to the desired end use of the patch 102. In some embodiments, the patch 102 can include more than one skin-contact adhesive 115. Where the patch 102 comprises more than one skin-contact adhesive layer 115, each skin-contact adhesive layer 115 may be selected independently of each other with regard to material and thickness used. Examples of suitable adhesives include acrylates, silicones, polyisobutylenes, synthetic rubber, natural rubber, and copolymers and mixtures thereof. Acrylates and silicones can be preferred skin-contact adhesives 115. In general, the skin-contact adhesive 115 should cause little or no irritation or sensitization of the skin during the intended wear period. Examples of skin-contact adhesives 115 that can be employed with the systems of the present disclosure include, but are not limited to, the adhesives described in U.S. Pat. Nos. RE24,906; 3,389,827; 6,103,369 and 4,499,896, which are incorporated herein by reference. In addition, silicone adhesives such as those described in U.S. Patent Publication No. 2011/0212325, which is incorporated herein by reference, can also be employed.

In some embodiments, e.g., in embodiments employing silicone adhesives, the patch 102 and the skin-contact adhesive 115 can be perforated to provide openings from the first major surface 110 of the patch 102 all the way through the second major surface 112 and the skin-contact adhesive 115, which can enhance permeability of the patch 102 and can minimize moisture build-up at the skin surface underlying the patch 102.

In some embodiments, the system 100 can further include one or more release liners over the skin-contact adhesive 115 (not shown) that can provide a release layer or surface to the skin-contact adhesive 115 prior to use. Examples of liners suitable for use with systems of the present disclosure can include, but are not limited to, kraft papers, polyethylene, polypropylene, polyester, or combinations thereof. Such liners can be coated with release agents, such as fluorochemicals, silicones, or other suitable low surface energy materials. Other adhesives and release liner combinations known to those of ordinary skill in the art can be employed in the systems of the present disclosure.

As mentioned above, the longitudinal flap 106 can be oriented to open and close substantially longitudinally (i.e., flaps longitudinally (i.e., distally) from its open position to its closed position) with respect to the longitudinal direction G of the securement system 100 and the medical article 60. The flap 106 can include a first major surface 120 and a second major surface 124, opposite the first major surface 120, the second major surface 124 configured to be coupled to at least a portion of the medical article 60 and the first major surface 110 of the patch 102 to secure the medical article 60 to the patch 102.

The second major surface 124 of the flap 106 can include coupling means, which can include one or more of a securing adhesive, a mechanical fastener (e.g., one mating surface of a mechanical fastener, with at least one of the medical article 60 and the patch 102 including the complementary mating surface), or a combination thereof. In some embodiments, the coupling means on the second major surface 124 of the flap 106 can include a securement assembly comprising a combination of adhesive and mechanical fastener, such as those disclosed in PCT Publication No. WO2014/014504, which is incorporated herein by reference.

In some embodiments, the coupling means on the second major surface 124 of the flap 106 can be repositionable to allow the flap 106 to be opened and closed multiple times while the system 100 remains on the patient. Similar classes of adhesives can be employed as a securing adhesive as described above with respect to the skin-contact adhesive 115. However, in some embodiments, if employed, the securing adhesive can have an adhesion that is higher than the skin-contact adhesive 115 on the patch 102.

In the embodiment of FIGS. 1A, 2 and 4A, the flap 106 includes a securing adhesive on at least a portion of its second major surface 124, and can be provided prior to use with one or more release liners 101, as shown in FIG. 1A. A two-sided butterfly-style release liner with side pull-tabs is shown by way of example only.

The flap 106 can further include a fixed proximal portion 130 (see FIGS. 2 and 4A) that is fixed with respect to the patch 102. The fixed proximal portion 130 can be coupled to the patch 102 and/or integrally formed with the patch 102. In embodiments in which the fixed proximal portion 130 is coupled to the patch 102, the fixed proximal portion 130 can be coupled to the patch 102 using a variety of coupling means including, but not limited to, one or more of adhesives, mechanical fasteners, cohesives, welding (e.g., sonic [e.g., ultrasonic] welding), any thermal bonding or heat sealing technique (e.g., heat and/or pressure applied to one or both of the components to be coupled), other suitable coupling means, or combinations thereof.

The flap 106 can further include a hinge (e.g., a living hinge) 132 (see FIGS. 2 and 4A) located within a perimeter 103 (see FIG. 2) of the patch 102, such that the hinge 132 does not extend to or form a portion of the perimeter 103 of the patch 102. As shown in FIG. 2, the hinge 132 can have a lateral width W in the lateral direction L (i.e., in a direction substantially perpendicular to the longitudinal direction G) that is less than a lateral width Y (i.e., a minimum lateral width Y) of the patch 102 and less than a lateral width X (i.e., a minimum lateral width X) of the flap 102 (i.e., when measured along the same direction, e.g., laterally, in the lateral direction L).

The narrow relative lateral width W of the hinge 132 can be important for ensuring that the hinge 132 can be located adjacent the multi-lumen joint 61 of the medical article 60 to provide a longitudinal stop to the medical article 60, e.g., a proximal stop to inhibit proximal pulling of the multi-lumen medical article 60. As shown in FIG. 2, the hinge 132 can be dimensioned to be received between two adjacent proximal tubings 62 of the medical article 60, such that it can provide at least a soft longitudinal stop to the medical article 60.

The narrow lateral width W of the hinge 132, relative to the flap 106 and the patch 102, can also ensure that while the hinge 132 is narrow, the remainder of the flap 106 and the patch 102 are sufficiently wide in the lateral direction L to effectively secure a portion (e.g., the multi-lumen joint 61) of the medical article 60 to the patch 102, and ultimately to the patient.

The flap 106 can further include a free distal portion 134 that is movable via the hinge 132 with respect to the patch 102 and the fixed proximal portion 130 of the flap 106 (and/or the medical article 60) between an open position (see FIGS. 1A and 4A) and a closed position (see FIG. 2).

Particularly, the free distal portion 134 can be movable longitudinally about a lateral hinge 132. In the open position, the medical article 60 is not secured by the flap 106. In the closed position, the medical article 60 is secured by the flap 106 to the patch 102 (i.e., the first major surface 110 thereof) to further inhibit at least normal movement (i.e., in addition to the longitudinal stop) of the medical article 60 relative to the patch 102.

The free distal portion 134 can be configured (e.g., shape, sized and positioned with respect to the patch 102) to overlap or cover at least the multi-lumen joint 61 of the medical article 60 and at least a portion of the patch to secure at least the multi-lumen joint 61 of the medical article 60 to the first major surface 110 of the patch 102 to inhibit at least normal movement of the medical article 60 relative to the patch 102.

In some embodiments, the system 100 can be configured to secure the medical article 60 in the region of one or more additional irregular features, in addition to the multi-lumen joint 61, to enhance securement of the medical article 60. That is, in some embodiments, the system 100 can be configured (e.g., one or more flaps 106 can be shaped, sized and/or positioned) to facilitate coupling to an irregular feature of the medical article 60, for example, by being coupled to a portion of the medical article 60 that comprises the irregular feature or by being coupled to a portion of the medical article 60 adjacent the irregular feature.

That is, some medical articles which can be secured by systems of the present disclosure can include irregular features over or adjacent which it can be useful to position at least a portion of one or more flaps 106. For example, an irregular feature of a medical article can include, but is not limited to, a multi-lumen joint (e.g., bifurcation point, trifurcation point, etc.), a change in diameter (e.g., a step-change or multi-step change), a protrusion (e.g., a knob, a dial, a meter, a connector), a constriction, or any other feature where a medical article may deviate from a uniform or regular shape, such as a tube or cylinder having a substantially uniform diameter.

The medical article 60 illustrates several examples of irregular features, including a protrusion (e.g., a radial protrusion) 65, a change in diameter 67 or 69, and the multi-lumen joint (e.g., a bifurcation) 61.

In some embodiments, the system 100 can include a plurality of hinges 132 (i.e., a plurality of flaps 106, each having a hinge 132). In such embodiments, each hinge 132 can be dimensioned to be received between two adjacent proximal tubings 62.

In some embodiments, the flap 106 can further include one or more proximal cutaway regions 140 located adjacent the fixed proximal portion 130 of the flap 106. Each proximal cutaway region 140 can extend from a perimeter of the flap 106 (e.g., from a proximal end of the flap 106) toward a central portion of the flap 106, without extending through the entire flap 106 to the opposite end. Each of the proximal cutaway regions 140 can be configured (e.g., sized and shaped) to receive a proximal tubing 62 of the medical article 60, and the hinge 132 can be located adjacent a proximal cutaway region 140 (see FIG. 2). Particularly, in some embodiments, as shown in FIGS. 1A and 2, the flap 106 can include two proximal cutaway regions 140, and the hinge 132 can be located between two adjacent proximal cutaway regions 140.

As shown in FIG. 1A, in some embodiments, each proximal cutaway region 140 can be defined at least partially by a longitudinally-extending, i.e., proximal, projection 146 of the flap 106. That is, each proximal cutaway region 140 can be shaped to define a portion of the flap 106 that is configured to be wrapped around at least a portion of a proximal tubing 62 of the multi-lumen medical article 60. As further shown in FIG. 1A, the system 100 includes three proximal projections 146 that define two proximal cutaway regions 140 therebetween.

By way of example, the specific configuration shown in FIG. 1A can be referred to as a diaper configuration, where the middle proximal projection 146 includes the fixed proximal portion 130 and the hinge 132 of the flap 106 and is dimensioned to be received between two adjacent proximal tubings 62, and the two outer proximal projections 146 are configured to be positioned (e.g., wrapped) around an outer lateral side of one of the proximal tubing 62. That is, each proximal projection 146 of the flap 106 can be configured to be secured around the outer lateral sides of the medical article 60, and optionally wrapped about (e.g., under) at least a portion of the medical article 60 before being secured to the first major surface 110 of the patch 102. As a result, employing one or more proximal projections 146 can further enhance wrapping and securing of a medical article and can further inhibit movement of the medical article 60 in one or more of the longitudinal direction G and the lateral direction L.

In such a configuration, the middle proximal projection 146 (i.e., the fixed proximal portion 130 and the hinge 132 of the flap 106) can be located substantially laterally centrally with respect to the flap 106, and each proximal cutaway region 140 can be located laterally off-axis or off-center with respect to an overall lateral width of the flap 106 or with respect to a central longitudinal axis of the flap 106. While the flap 106 of the system 100 is substantially laterally centered with respect to the patch 102, it should be understood that this need not be the case.

In some embodiments, the flap 106 can include fewer or more than two proximal cutaway regions 140 and fewer or more than three proximal projections. In such embodiments, the system 100 can still include (i) a first proximal projection 146 of the flap 106 that includes the fixed proximal portion 130 and the hinge 132 of the flap 106 and can optionally further include (ii) a second proximal projection 146 that can be shaped to be positioned around (e.g., and optionally under) at least a portion of the medical article 60 (e.g., a proximal tubing 62) when the flap 106 is secured to the medical article 60 and the patch 102.

In some embodiments, the flap 106 can further include one or more distal cutaway regions 142 (see FIGS. 1A and 2) located in the free distal portion 134 of the flap 106. Such distal cutaway regions can extend from a perimeter of the flap 106 (e.g., from a distal end of the flap 106) toward a central portion of the flap, without extending through the entire flap 106 to the opposite end. Each distal cutaway region 140 can be dimensioned to receive a distal tubing 64 of the medical article 60. In some embodiments, the flap 106 can include a distal cutaway region 142 located substantially centrally with respect to the lateral width of the flap 106 or with respect to a central longitudinal axis of the flap 106.

By way of example only, each of the proximal cutaway regions 140 of the system 100 has a rounded, smooth, upside-down “U” shape, and the distal cutaway region 142 had a V-notch shape. However, these shapes are shown by way of example only, and the proximal cutaway regions 140 and the distal cutaway region 142 can have any shape necessary to facilitate coupling the flap 106 to the medical article 60 and further to the first major surface 110 of the patch 102.

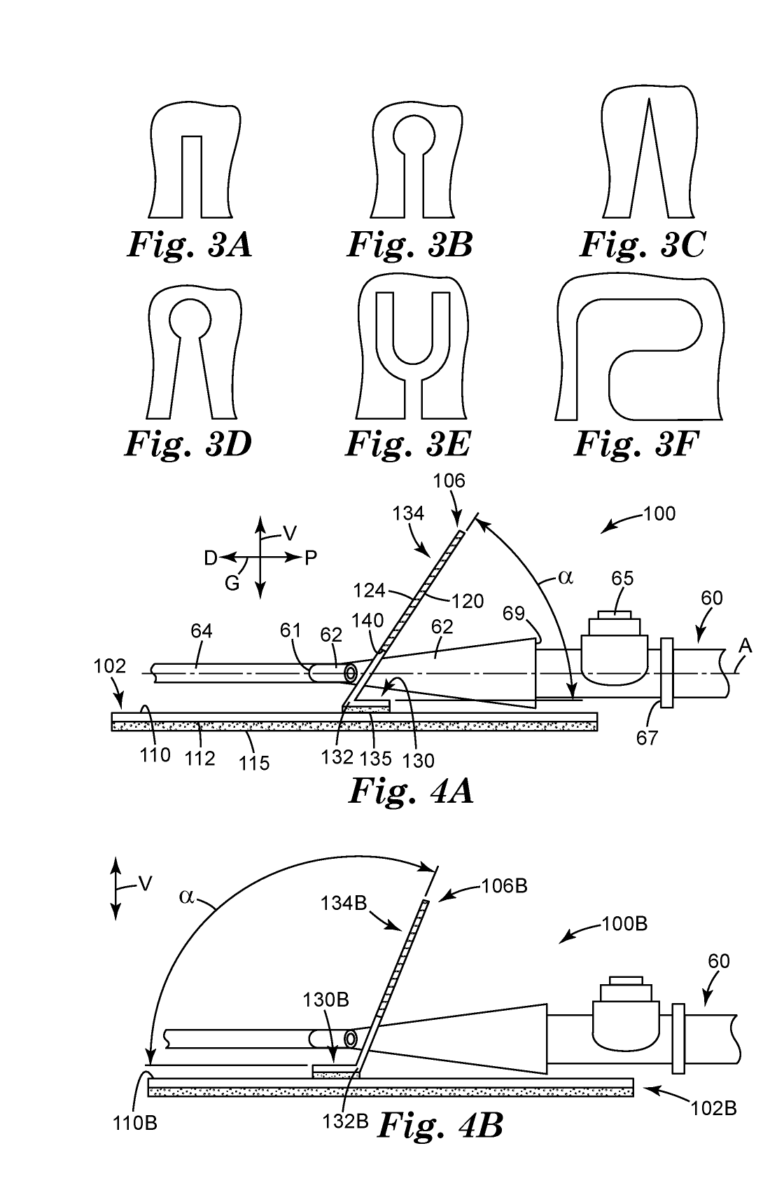

FIGS. 3A-3F illustrate a variety of shapes that can be employed as one or more of the proximal cutaway regions 140 or distal cutaway regions 142 of the flap 106 of the present disclosure. FIG. 3A shows a cutaway region having a straight channel (e.g., rectangular) shape. FIG. 3B shows a cutaway region having the shape of a straight channel with a circular end, i.e., a rectangular-circular combination shape. FIG. 3C shows a cutaway region with a triangular shape. FIG. 3D shows a cutaway regions with a key-hole shape, i.e., a frusto-triangular channel with a circular end. FIG. 3E shows a cutaway region with a forked shape. FIGS. 3A-3E all illustrate symmetrical cutaway region shapes, however, in some embodiments, asymmetrical shapes can be employed. By way of example, FIG. 3F shows a cutaway region having an asymmetrical L-shape. FIGS. 3E and 3F are also examples of non-linear or irregular shapes.

As a result, in some embodiments, one or more of the cutaway regions is not merely a slit, but rather is a larger opening dimensioned to accommodate at least a portion of a proximal tubing of a multi-lumen medical article. Such cutaway regions can include a variety of shapes, including, but not limited to, one or more of rectangular, triangular, rectangular-circular combination, triangular, key-hole shaped, forked or fork-shaped (e.g., with a U, V or Y shaped fork), L-shaped, other suitable shapes, or a combination thereof.

As shown in FIGS. 1A, 2 and 4A, in some embodiments, a central portion of the flap 106 (i.e., a longitudinally central portion) can be free of any cutaway regions and can be configured to cover (and secure) a portion of the medical article 60 (e.g., an upper surface thereof) comprising the multi-lumen joint 61.

As mentioned above, in some embodiments, the system 100 can be free of any rigid components, such as rigid securement devices, that are more rigid than the flap 106. Examples of rigid components, and particularly, rigid securement devices, can include, but are not limited to, one or more of brackets, retainers, clips, posts, clamps, hooks, other typical rigid devices or structures, or a combination thereof.

However, FIG. 1B shows an alternative system 100A according to one embodiment of the present disclosure. The system 100A of FIG. 1B is identical to the system 100 described above and shown in FIGS. 1A, 2 and 4A, except that the system 100A further includes a post 150 that is more rigid than the flap 106 and that can be coupled (directly or indirectly) to the first major surface 110 of the patch 102 and located adjacent the hinge 132, such that the post 150 is configured to provide, or assist the hinge 132 in providing, a longitudinal stop for the medical article 60. As a result, similar to the hinge 132, the post 150, if employed, can be dimensioned to be received between two adjacent proximal tubings 62 of the medical article 60. In some embodiments, the post 150 can be formed of a rigid plastic, a soft plastic, and/or a foam, while still being more rigid than the flap 106 and the patch 102.

As mentioned above, the fixed proximal portion 130 of the flap 106 can be coupled to the patch 102 via a variety of coupling means. FIG. 4A shows one example of this in which a securing adhesive 135 is used to couple the second major surface 124 of the fixed proximal portion 130 of the flap 106 to the first major surface 110 of the patch 102. In such embodiments, the securing adhesive 135 can be the same adhesive as that used in the free distal portion 134 to secure the medical article 60, or can be a different adhesive. For example, while not illustrated in FIG. 4A, in some embodiments, the securing adhesive 135 can be present over the second major surface 124 of the flap 106, in the fixed proximal portion 130 and the free distal portion 134. In such embodiments, the securing adhesive 135 can be coextensive with the second major surface 124 of the flap 106 but need not be.

In some embodiments, the first major surface 110 of the patch 102, or a portion thereof to which the free distal portion 134 of the flap 106 will be coupled to secure the medical article 60 can include a low adhesion backsize coating to function as a release layer for the free distal portion 134, i.e., when a securing adhesive is employed on the second major surface 124 of the free distal portion 134 of the flap 106.

As shown in FIGS. 2 and 4A, in some embodiments, the fixed proximal portion 130, the hinge 132, and the free distal portion 134 of the flap 106 can be arranged such that the fixed proximal portion 140 is located proximally with respect to the hinge 132. In such embodiments, the fixed proximal portion 130 and the free distal portion 134 can be separated by an angle α (see FIG. 4A) that increases as the free distal portion 134 moves from the open position to the closed position. That is, in some embodiments, the angle α can be less than or equal to 90 degrees when the free distal portion 134 is in the open position, and the angle α can be obtuse when the free distal portion 134 is in the closed position. Furthermore, in such embodiments, the fixed proximal portion 130, the hinge 132, and the free distal portion 134 of the flap 106 can be arranged such that the free distal portion 134 of the flap 106 does not overlap the fixed proximal portion 130 of the flap 106 when the flap 106 is in the closed position.

As a result, in embodiments in which the fixed proximal portion 130 of the flap 106 is located proximally with respect to the hinge 132 (see FIG. 4A), any displacement or decoupling force exerted on the medical article 60 in the vertical direction V (e.g., by a vertical pulling of a proximal end of the medical article 60), would initiate a decoupling (i.e., disengagement) of the fixed proximal portion 130 from the first major surface 110 of the patch 102 predominantly in tensile mode. Such an arrangement of the flap 106 can be particularly advantageous when coupling means are employed between the fixed proximal portion 130 of the flap 106 and the patch 102 that have a greater tensile strength than peel strength. The resistance to decoupling, e.g., for an adhesive or a mechanical fastener, is generally higher in tensile mode than in peel mode. As a result, the configuration shown in FIG. 4A (as compared to FIG. 4B shown below) can be particularly advantageous for to coupling the fixed proximal portion 130 of the flap 106 to the patch 102.

However, other configurations can be employed. FIG. 4B illustrates an alternative system 100B according to one embodiment of the present disclosure. The system 100B is identical to the system 100, except that the fixed proximal portion 130B of the flap 106B is located distally with respect to the hinge 132B (i.e., such that the flap 106B is folded back on itself longitudinally when in the closed position). In such embodiments, the fixed proximal portion 130B of the flap 106B is configured to be positioned under at least a portion of the medical article 60 (i.e., such that the flap 106B wraps around a lower surface of the medical article 60 and an upper surface of the medical article 60, and at least a portion of the medical article 60 is captured between the fixed proximal portion 130B and the free distal portion 134B of the flap 106B).

Furthermore, in such embodiments, the fixed proximal portion 130B and the free distal portion 134B are separated by an angle α and arranged such that the angle α decreases as the free distal portion 134B moves from the open position to the closed position. That is, in some embodiments, the angle α can be obtuse when the free distal portion 134B is in the open position, and the angle α can be less than or equal to 90 degrees when the free distal portion 134B is in the closed position.

Furthermore, in such embodiments, as shown in FIG. 4B, the fixed proximal portion 130B, the hinge 132B, and the free distal portion 134B are arranged such that at least a portion of the free distal portion 134B of the flap 106B overlaps at least a portion of the fixed proximal portion 130B of the flap 106B when the flap is in the closed position, and such that at least a portion of the medical article 60 is located between (e.g., sandwiched between) the fixed proximal portion 130B and the free distal portion 134B of the flap 106B when secured.

As a result, in embodiments in which the fixed proximal portion 130B of the flap 106B is located distally with respect to the hinge 132B (see FIG. 4B), any displacement or decoupling force exerted on the medical article 60 in the vertical direction V (e.g., by a vertical pulling of a proximal end of the medical article 60), would initiate a decoupling (i.e., disengagement) of the fixed proximal portion 130B from the first major surface 110B of the patch 102B predominantly in peel mode.

### Backings

Suitable backings for patches and/or flaps of the present disclosure can include, but are not limited to, one or more of a fabric, a woven fibrous web, a nonwoven fibrous web, a knit, a polymeric film, other familiar dressing materials, or combinations thereof. In some embodiments, the backing materials can include polymeric elastic films (e.g., transparent or non-transparent), and can include, but are not limited to, films formed of elastomeric polyurethanes, co-polyesters, polyethylenes, or combinations thereof. The backing can be a high moisture vapor permeable film, i.e., a backing with a relatively high moisture vapor transmission rate (MVTR). U.S. Pat. No. 3,645,835 describes methods of making such films and methods for testing their permeability. The backing can be constituted of natural or synthetic sources of raw materials.

In some embodiments, the backing (e.g., for a patch of the present disclosure) can include a support device with a contained cushion element, such as those disclosed in PCT Publication No. WO2015/020875, which is incorporated herein by reference.

The backings of patches of the present disclosure advantageously should transmit moisture vapor at a rate equal to or greater than human skin. In some embodiments, the patch backing can be adhesive-coated. In such embodiments, the adhesive-coated backing can transmit moisture vapor at a rate of at least 300 g/m²/24 hrs/37° C./100-10% RH, and in some embodiments, at least 700 g/m²/24 hrs/37° C./100-10% RH. The patch backing is generally conformable to anatomical surfaces. As such, when the patch is applied to an anatomical surface, it conforms to the surface even when the surface is moved.

The backing of patches and/or flaps of the present disclosure can be a flexible material. For example, the backing can be a film, paper, woven, knit, foam, nonwoven material, or a combination thereof, or one or more layers of film, paper, woven, knit, foam, nonwoven, or a combination thereof. In some embodiments, it can be desirable that at least a portion of backing is formed of a transparent material to allow for viewing of underlying skin, a medical device, and/or a target site.

By way of example only, in some embodiments, the backing of a patch of the present disclosure can be formed of a film available under the trade designation TEGADERM® from 3M Company, St. Paul, Minn.

Each embodiment shown in the figures is illustrated as a separate embodiment for clarity in illustrating a variety of features of the securement systems of the present disclosure. However, it should be understood that any combination of elements and features of any of the embodiments illustrated in the figures and described herein can be employed in the securement systems of the present disclosure.

The following embodiments are intended to be illustrative of the present disclosure and not limiting.

### Embodiments

Embodiment 1 is a securement system for securing a multi-lumen medical article, the medical article comprising a multi-lumen joint, at least two proximal tubings connected to the multi-lumen joint and at least one distal tubing connected to the multi-lumen joint, the system comprising:
a longitudinal direction;
a patch comprising
   a first major surface configured to receive at least a portion of the medical article, and
   a second major surface, opposite the first major surface, comprising a skin-contact adhesive; and
a longitudinal flap comprising
   a first major surface,
   a second major surface, opposite the first major surface, configured to be coupled to at least a portion of the medical article and the first major surface of the patch,
   a fixed proximal portion that is fixed with respect to the patch,
   a hinge located within a perimeter of the patch, such that the hinge does not extend to or form a portion of the perimeter of the patch, the hinge having a lateral width that is less than a lateral width of the patch and less than a lateral width of the flap, wherein the hinge is dimensioned to be received between two adjacent proximal tubings of the medical article, and
   a free distal portion that is movable via the hinge with respect to the patch and the fixed proximal portion of the flap between an open position and a closed position, the free distal portion being configured to overlap at least the multi-lumen joint of the medical article and at least a portion of the patch to secure at least the multi-lumen joint of the medical article to the first major surface of the patch.

Embodiment 2 is the system of embodiment 1, wherein the hinge provides a longitudinal stop to the multi-lumen medical article.

Embodiment 3 is the system of embodiment 1 or 2, wherein the flap is one of a plurality of flaps, and wherein each hinge is dimensioned to be received between two adjacent proximal tubings.

Embodiment 4 is the system of any of embodiments 1-3, wherein the flap further includes at least two proximal cutaway regions located adjacent the fixed proximal portion of the flap, each proximal cutaway region extending from a perimeter of the flap toward a central portion of the flap, wherein each of the proximal cutaway regions is configured to receive a proximal tubing of the medical article, and wherein the hinge is located between two adjacent proximal cutaway regions.

Embodiment 5 is the system of embodiment 4, wherein each proximal cutaway region is defined at least partially by two proximal projections of the flap, and wherein the two proximal projections are configured to straddle a proximal tubing.

Embodiment 6 is the system of embodiment 5, wherein at least one proximal projection includes the fixed proximal portion and the hinge of the flap.

Embodiment 7 is the system of any of embodiments 1-3, wherein the flap includes at least one proximal cutaway region extending from a perimeter of the flap toward a central portion of the flap, wherein the proximal cutaway region is configured to receive a proximal tubing of the medical article, and wherein each proximal cutaway region is defined at least partially by a proximal projection of the flap.

Embodiment 8 is the system of embodiment 7, wherein the proximal projection includes the fixed proximal portion and the hinge of the flap.

Embodiment 9 is the system of embodiment 7 or 8, wherein the flap includes two proximal cutaway regions and three proximal projections, and wherein the middle proximal projection includes the fixed proximal portion and the hinge of the flap.

Embodiment 10 is the system of embodiment 9, wherein the hinge is located centrally with respect to the lateral width of the flap, and wherein each proximal cutaway region is located off-center with respect to the lateral width of the flap.

Embodiment 11 is the system of any of embodiments 7-10, wherein at least one proximal cutaway region is defined by (i) a first proximal projection of the flap that includes the fixed proximal portion and the hinge of the flap and (ii) a second proximal projection configured to be wrapped around at least a portion of the medical article when the flap is secured to the medical article.

Embodiment 12 is the system of any of embodiments 4-11, wherein at least one proximal cutaway region is key-hole shaped, rectangular, triangular, forked, L-shaped, or a combination thereof.

Embodiment 13 is the system of any of embodiments 4-12, wherein a central portion of the flap is free of any cutaway regions and is configured to cover at least a portion of the multi-lumen joint of the medical article.

Embodiment 14 is the system of any of embodiments 1-13, wherein the flap includes at least one distal cutaway region located in the free distal portion of the flap, the at least one distal cutaway region extending from a perimeter of the flap toward a central portion of the flap, wherein the at least one distal cutaway region is dimensioned to receive a distal tubing of the medical article.

Embodiment 15 is the system of embodiment 14, wherein the at least one distal cutaway region is located substantially centrally with respect to the lateral width of the flap.

Embodiment 16 is the system of any of embodiments 1-15, wherein the system is free of a rigid component that is more rigid than the flap.

Embodiment 17 is the system of any of embodiments 1-16, further comprising a post coupled to the first major surface of the patch and located adjacent the hinge, such that the post is configured to provide a longitudinal stop for the medical article and such that at least a portion of the post is dimensioned to be received between two adjacent proximal tubings of the medical article.

Embodiment 18 is the system of any of embodiments 1-17, wherein the flap is integrally formed with the patch.

Embodiment 19 is the system of any of embodiments 1-18, wherein the fixed proximal portion of the flap is located distally with respect to the hinge of the flap.

Embodiment 20 is the system of any of embodiments 1-19, wherein the fixed proximal portion, the hinge, and the free distal portion of the flap are arranged such that at least a portion of the fixed proximal portion of the flap is positioned under at least a portion of the medical article, when a medical article is coupled to the system.

Embodiment 21 is the system of any of embodiments 1-20, wherein the fixed proximal portion and the free distal portion are separated by an angle and are arranged such that the angle decreases as the free distal portion moves from the open position to the closed position.

Embodiment 22 is the system of any of embodiments 1-21, wherein the fixed proximal portion, the hinge, and the free distal portion are arranged such that at least a portion of the free distal portion of the flap overlaps at least a portion of the fixed proximal portion of the flap when in the closed position.

Embodiment 23 is the system of any of embodiments 1-18, wherein the fixed proximal portion of the flap is located proximally with respect to the hinge of the flap.

Embodiment 24 is the system of any of embodiments 1-18 and 23, wherein the fixed proximal portion and the free distal portion are separated by an angle and are arranged such that the angle increases as the free distal portion moves from the open position to the closed position.

Embodiment 25 is the system of any of embodiments 1-18 and 23-24, wherein the fixed proximal portion, the hinge, and the free distal portion of the flap are arranged such that the free distal portion of the flap does not overlap the fixed proximal portion of the flap when in the closed position.

Embodiment 26 is the system of any of embodiments 1-25, wherein the first major surface of the patch includes a low adhesion backsize coating.

It is to be understood that the invention is not limited in its application to the details of construction and the arrangement of components set forth in the above description or illustrated in the accompanying drawings. The invention is capable of other embodiments and of being practiced or of being carried out in various ways. Also, it is to be understood that the phraseology and terminology used herein is for the purpose of description and should not be regarded as limiting. It is to be further understood that other embodiments may be utilized, and structural or logical changes may be made without departing from the scope of the present disclosure.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

Various features and aspects of the present disclosure are set forth in the following claims.

1. A securement system for securing a multi-lumen medical article, the medical article comprising a multi-lumen joint, at least two proximal tubings connected to the multi-lumen joint and at least one distal tubing connected to the multi-lumen joint, the system comprising:
a longitudinal direction;
a patch comprising
a first major surface configured to receive at least a portion of the medical article, and
a second major surface, opposite the first major surface, comprising a skin-contact adhesive; and
a longitudinal flap comprising
a first major surface,
a second major surface, opposite the first major surface, configured to be coupled to at least a portion of the medical article and the first major surface of the patch,
a fixed proximal portion that is fixed with respect to the patch,
a hinge located within a perimeter of the patch, such that the hinge does not extend to or form a portion of the perimeter of the patch, the hinge having a lateral width that is less than a lateral width of the patch and less than a lateral width of the flap, wherein the hinge is dimensioned to be received between two adjacent proximal tubings of the medical article, and
a free distal portion that is movable via the hinge with respect to the patch and the fixed proximal portion of the flap between an open position and a closed position, the free distal portion being configured to secure at least the multi-lumen joint of the medical article to the first major surface of the patch.

2. The system of claim 1, wherein the hinge provides a longitudinal stop to the multi-lumen medical article.

3. The system of claim 1, wherein the flap further includes at least two proximal cutaway regions located adjacent the fixed proximal portion of the flap, each proximal cutaway region extending from a perimeter of the flap toward a central portion of the flap, wherein each of the proximal cutaway regions is configured to receive a proximal tubing of the medical article, and wherein the hinge is located between two adjacent proximal cutaway regions.

4. The system of claim 1, wherein the flap includes at least one proximal cutaway region extending from a perimeter of the flap toward a central portion of the flap, wherein the proximal cutaway region is configured to receive a proximal tubing of the medical article, and wherein each proximal cutaway region is defined at least partially by a proximal projection of the flap.

5. The system of claim 4, wherein the proximal projection includes the fixed proximal portion and the hinge of the flap.

6. The system of claim 4, wherein the flap includes two proximal cutaway regions and three proximal projections, and wherein the middle proximal projection includes the fixed proximal portion and the hinge of the flap.

7. The system of claim 4, wherein at least one proximal cutaway region is defined by (i) a first proximal projection of the flap that includes the fixed proximal portion and the hinge of the flap and (ii) a second proximal projection configured to be wrapped around at least a portion of the medical article when the flap is secured to the medical article.

8. The system of claim 4, wherein at least one proximal cutaway region is key-hole shaped, rectangular, triangular, forked, L-shaped, or a combination thereof.

9. The system of claim 1, wherein the flap includes at least one distal cutaway region located in the free distal portion of the flap, the at least one distal cutaway region extending from a perimeter of the flap toward a central portion of the flap, wherein the at least one distal cutaway region is dimensioned to receive a distal tubing of the medical article.

10. The system of claim 1, wherein the system is free of a rigid component that is more rigid than the flap.

11. The system of claim 1, further comprising a post coupled to the first major surface of the patch and located adjacent the hinge, such that the post is configured to provide a longitudinal stop for the medical article and such that at least a portion of the post is dimensioned to be received between two adjacent proximal tubings of the medical article.

12. The system of claim 1, wherein the flap is integrally formed with the patch.

13. The system of claim 1, wherein the fixed proximal portion of the flap is located distally with respect to the hinge of the flap.

14. The system of claim 1, wherein the fixed proximal portion and the free distal portion are separated by an angle and are arranged such that the angle decreases as the free distal portion moves from the open position to the closed position.

15. The system of claim 1, wherein the fixed proximal portion of the flap is located proximally with respect to the hinge of the flap.

16. The system of claim 2, wherein the flap further includes at least two proximal cutaway regions located adjacent the fixed proximal portion of the flap, each proximal cutaway region extending from a perimeter of the flap toward a central portion of the flap, wherein each of the proximal cutaway regions is configured to receive a proximal tubing of the medical article, and wherein the hinge is located between two adjacent proximal cutaway regions.

17. The system of claim 2, wherein the flap includes at least one proximal cutaway region extending from a perimeter of the flap toward a central portion of the flap, wherein the proximal cutaway region is configured to receive a proximal tubing of the medical article, and wherein each proximal cutaway region is defined at least partially by a proximal projection of the flap.

18. The system of claim 3, wherein the flap includes at least one proximal cutaway region extending from a perimeter of the flap toward a central portion of the flap, wherein the proximal cutaway region is configured to receive a proximal tubing of the medical article, and wherein each proximal cutaway region is defined at least partially by a proximal projection of the flap.

19. The system of claim 5, wherein the flap includes two proximal cutaway regions and three proximal projections, and wherein the middle proximal projection includes the fixed proximal portion and the hinge of the flap.

20. The system of claim 5, wherein at least one proximal cutaway region is defined by (i) a first proximal projection of the flap that includes the fixed proximal portion and the hinge of the flap and (ii) a second proximal projection configured to be wrapped around at least a portion of the medical article when the flap is secured to the medical article.
